(19) United States
(12) Patent Application Publication
(10) Pub. No.: US 2006/0211700 A1
(43) Pub. Date: Sep. 21, 2006

(54) (R)-8,9-dichloro-2,3,4,4a-tetrahydro-1H,6H-pyrazino[1,2-a]quinoxalin-5-one for controlling IOP and treating glaucoma

(75) Inventors: Jesse A. May, Fort Worth, TX (US); Paul W. Zinke, Fort Worth, TX (US)

Correspondence Address: Alcon Research, Ltd.;Patrick M. Ryan(Q-148), IP Legal Department, 6201 So. Freeway, Fort Worth, TX, 76134-2099, US

(73) Assignee: Alcon, Inc.

(21) Appl. No.: 11/385,187

(22) Filed: Mar. 21, 2006

Related U.S. Application Data
(60) Provisional application No. 60/663,803, filed on Mar. 21, 2005.

(51) Int. Cl. A61K 31/498

(52) U.S. Cl. 514/250

(57) ABSTRACT

Compositions and methods for controlling intraocular pressure and treating glaucoma using (R)-8,9-dichloro-2,3,4,4a-tetrahydro-1H,6H-pyrazino[1,2-a]quinoxalin-5-one are disclosed.

This application claims priority to U.S. Provisional Application, U.S. Ser. No. 60/663,803 filed Mar. 21, 2005.

The present invention is directed to the use of (R)-8,9-dichloro-2,3,4,4a-tetrahydro-1H,6H-pyrazino[1,2-a]quinoxalin-5-one for lowering and controlling intraocular pressure (IOP) and treating glaucoma.

### BACKGROUND OF THE INVENTION

The disease state referred to as glaucoma is characterized by a permanent loss of visual function due to irreversible damage to the optic nerve. The several morphologically or functionally distinct types of glaucoma are typically characterized by elevated IOP, which is considered to be causally related to the pathological course of the disease. Ocular hypertension is a condition wherein intraocular pressure is elevated but no apparent loss of visual function has occurred; such patients are considered to be a high risk for the eventual development of the visual loss associated with glaucoma. Some patients with glaucomatous field loss have relatively low intraocular pressures. These so called normotension or low tension glaucoma patients can also benefit from agents that lower and control IOP. If glaucoma or ocular hypertension is detected early and treated promptly with medications that effectively reduce elevated intraocular pressure, loss of visual function or its progressive deterioration can generally be ameliorated. Drug therapies that have proven to be effective for the reduction of intraocular pressure include both agents that decrease aqueous humor production and agents that increase the outflow facility. Such therapies are in general administered by one of two possible routes, topically (direct application to the eye) or orally.

There are some individuals who do not respond well when treated with certain existing glaucoma therapies. There is, therefore, a need for other topical therapeutic agents that control IOP.

Certain pyrazino[1,2-a]quinoxaline compounds have been disclosed as having utility in the treatment of a variety of CNS disorders, including obsessive-compulsive disorder, anxiety, panic disorder, schizophrenia, migraine, sleep disorders, eating disorders, obesity, type II diabetes, and epilepsy. These utility claims are predicated on the observation that these compounds function primarily as agonists at the human 5-HT_{2c} receptor. See WO 00/35922, WO 02/59127, and Biorg. Med. Chem. Lett., 10:1991 (2000). Though high affinity at the human 5-HT_{2C} receptor has been reported for selected pyrazino[1,2-a]quinoxalines, the affinity for these compounds at human 5HT_{2A} or 5-HT_{2B} receptors has not been reported. Furthermore, these compounds have not been assayed against any of the 5-HT₂ receptors isolated from any other species, e.g., porcine or bovine. Additionally, certain other pyrazino[1,2-a]quinoxaline derivatives have previously been disclosed as systemic hypotensive agents for the treatment of hypertension. See U.S. Pat. No. 4,032,639, U.S. Pat. No. 4,089,958, and Indian J. Chemistry, 17B:244 (1979). None of the references cited above associate any of the reported or claimed pyrazino[1,2-a]quinolaxines with utility for the treatment of ocular hypertension or glaucoma. U.S. Pat. No. 6,664,286 discloses the use of 5-HT₂ agonists for the treatment of ocular hypertension and glaucoma, but it does not disclose the pyrazino[1,2-a]quinoxaline derivative of the present invention.

### SUMMARY OF THE INVENTION

The present invention is directed to compositions of (R)-8,9-dichloro-2,3,4,4a-tetrahydro-1H,6H-pyrazino[1,2-a]quinoxalin-5-one and its use for lowering and controlling IOP and treating glaucoma.

### DESCRIPTION OF PREFERRED EMBODIMENTS

8,9-dichloro-2,3,4,4a-tetrahydro-1H,6H-pyrazino[1,2-a]quinoxalin-5-one is a known compound. It is also known as “WAY-161503” and can be made by methods disclosed in WO 00/35922, which is incorporated by reference herein. It is commercially available (Tocris).

8,9-dichloro-2,3,4,4a-tetrahydro-1H,6H-pyrazino[1,2-a]quinoxalin-5-one is a racemic compound. The R enantiomer (“Compound”) has been reported to be approximately 40-fold more potent than the S enantiomer at the 5-HT_{2C} receptor. No data has been reported for the activity of these enantiomers at either the 5-HT_{2A} or the 5-HT_{2B} receptors. The present invention encompasses the use of the R enantiomer substantially free of the S enantiomer. One enantiomer is “substantially free” of the other enantiomer if less than 2% of the unwanted enantiomer is present.

The Compound can be incorporated into various types of ophthalmic formulations for delivery to the eye (e.g., topically, intracamerally, or via an implant). It is preferably incorporated into topical ophthalmic formulations for delivery to the eye. The Compound may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving the Compound in a physiologically acceptable isotonic aqueous buffer. Further, the ophthalmic solution may include an ophthalmologically acceptable surfactant to assist in dissolving the Compound. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan gum and xanthan gum. In order to prepare sterile ophthalmic ointment formulations, the Compound is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-974, or the like, according to the published formulations for analogous ophthalmic preparations; preservatives and tonicity agents can be incorporated.

The Compound is preferably formulated as a topical ophthalmic suspension or solution, with a pH of about 5 to 8. The Compound will normally be contained in these formulations in an amount 0.01% to 5% (w/v), but preferably in an amount of 0.25% to 2% (w/v). Thus, for topical presentation 1 to 2 drops of these formulations would be delivered to the surface of the eye 1 to 4 times per day according to the discretion of a skilled clinician.

The Compound can also be used in combination with other agents for treating glaucoma, such as, but not limited to, β-blockers, prostaglandins, carbonic anhydrase inhibitors, α2 agonists, miotics, and neuroprotectants.

The following topical ophthalmic formulations are useful according to the present invention administered 1-4 times per day according to the discretion of a skilled clinician.

### Example 1

| Ingredients | Amount (wt %) |
|---|---|
| Compound | 0.01-2% |
| Hydroxypropyl methylcellulose | 0.5% |
| Dibasic sodium phosphate | 0.2% |
| (anhydrous) | |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | q.s. to pH to 7.3-7.4 |
| Purified water | q.s. to 100% |

### Example 2

| Ingredients | Amount (wt %) |
|---|---|
| Compound | 0.01-2% |
| Methyl cellulose | 4.0% |
| Dibasic sodium phosphate | 0.2% |
| (anhydrous) | |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | q.s. to pH to 7.3-7.4 |
| Purified water | q.s. to 100% |

### Example 3

| Ingredients | Amount (wt %) |
|---|---|
| Compound | 0.01-2% |
| Guar gum | 0.4-6.0% |
| Dibasic sodium phosphate | 0.2% |
| (anhydrous) | |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | q.s. to pH to 7.3-7.4 |
| Purified water | q.s. to 100% |

### Example 4

| Ingredients | Amount (wt %) |
|---|---|
| Compound | 0.01-2% |
| White petrolatum and mineral oil and | Ointment consistency |
| lanolin | |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | q.s. to pH to 7.3-7.4 |

The ability of the Compound to lower IOP was evaluated.

### Example 5

Acute IOP Response in Lasered (Hypertensive) Eyes of Conscious Cynomolgus Monkeys

Intraocular pressure (IOP) can be determined with an Alcon Pneumatonometer after light corneal anesthesia with 0.1% proparacaine. Eyes are washed with saline after each measurement. After a baseline IOP measurement, test compound is instilled in one 30 μL aliquot to the right eyes only of nine cynomolgus monkeys. Vehicle is instilled in the right eyes of six additional animals. Subsequent IOP measurements are taken at 1, 3, and 6 hours.

The above method was used to determine the IOP lowering efficacy of 8,9-dichloro-2,3,4,4a-tetrahydro-1H,6H-pyrazino[1,2-a]quinoxalin-5-one when dosed at 300 μg. The results shown in the following table demonstrate that 8,9-dichloro-2,3,4,4a-tetrahydro-1H,6H-pyrazino[1,2-a]quinoxalin-5-one caused a significant decrease in IOP.

| | Compound | | Vehicle Control | |
|---|---|---|---|---|
| | IOP Change | | IOP Change | IOP % |
| Time (hrs.) | mmHg | IOP % change | mmHg | Change |
| 1 | −2.8 | −5.7 | −1.5 | −3.4 |
| 3 | −9.6 | −21.9 | −4.0 | −9.5 |
| 6 | −12.8 | −29.3 | −3.8 | −8.9 |

The same method was used to evaluate the IOP lowering ability of the R enantiomer of the Compound (substantially free of the S enantiomer), dosed at 300 μg. The results shown in the following table demonstrate that the R enantiomer caused a significant decrease in IOP.

| | R - Enantiomer of Compound | | Vehicle Control | |
|---|---|---|---|---|
| | IOP Change | | IOP Change | IOP % |
| Time (hrs.) | mmHg | IOP % change | mmHg | Change |
| 1 | −5.8 | −12.9 | −0.3 | −0.3 |
| 3 | −11.7 | −26.3 | −2.6 | −5.9 |
| 6 | −15.0 | −34.7 | −6.0 | −14.0 |

### Example 6

In Vitro Functional Response at 5-HT₂ Receptors

Functional response at the 5-HT₂ receptor subtypes was determined using CHO-K1 cells stably expressing mitochondrially-targeted bioluminescent aequorin, G_{α16}, and one of either human serotonin receptor clone 5-HT_{2A}, 5-HT_{2B}, or 5-HT_{2C}. Prior to testing, cells were loaded in suspension with coelenterazine for 4-16 hours and directly injected onto different concentrations of the test compound. Light emitted from the cells was measured 20-30 seconds following receptor activation. A luminometer was used to record luminescence in response to the test compound. The mean response signal at each of 8-11 different concentrations was integrated to provide an estimation of receptor activation, expressed as the EC₅₀ value. The efficacy of the response (Eₘₐₓ) at the 5-HT_{2A} and 5-HT_{2B} receptors is expressed relative to the response of α-methyl-5-HT under the same assay conditions while the efficacy at 5-HT_{2C} is expressed relative to the response of 5-HT.

| | 5-HT_{2A} | | 5-HT_{2B} | | 5-HT_{2C} | |
|---|---|---|---|---|---|---|
| | EC₅₀ | | EC₅₀ | | EC₅₀ | |
| | nM | Eₘₐₓ % | nM | Eₘₐₓ % | nM | Eₘₐₓ % |
| Racemic | 37.5 | 112 | 76.8 | 98 | 4.2 | 105 |
| R enantiomer | 20.5 | 108 | 31.6 | 99 | 2.6 | 108 |
| S enantiomer | 2260 | 96 | 2540 | 45 | 614 | 105 |

We claim:

1. A method for controlling intraocular pressure in an eye of a patient which comprises administering to the patient a composition comprising a pharmaceutically acceptable carrier and a pharmaceutically effective amount of (R)-8,9-dichloro-2,3,4,4a-tetrahydro-1H,6H-pyrazino[1,2-a]quinoxalin-5-one substantially free of (S)-8,9-dichloro-2,3,4,4a-tetrahydro-1H,6H-pyrazino[1,2-a]quinoxalin-5-one.

2. The method of claim 1 wherein the composition is topically administered to the eye of the patient.

3. The method of claim 2 wherein the pharmaceutically effective amount is 0.01% to 5% (w/v).

4. The method of claim 3 wherein the pharmaceutically effective amount is 0.25% to 2% (w/v).

5. A method for treating glaucoma in an eye of a patient which comprises administering to the patient a composition comprising a pharmaceutically acceptable carrier and a pharmaceutically effective amount of (R)-8,9-dichloro-2,3,4,4a-tetrahydro-1H,6H-pyrazino[1,2-a]quinoxalin-5-one substantially free of (S)-8,9-dichloro-2,3,4,4a-tetrahydro-1H,6H-pyrazino[1,2-a]quinoxalin-5-one.

6. The method of claim 5 wherein the composition is topically administered to the eye of the patient.

7. The method of claim 6 wherein the pharmaceutically effective amount is 0.01% to 5% (w/v).

8. The method of claim 7 wherein the pharmaceutically effective amount is 0.25% to 2% (w/v).
